(19) United States
(12) Patent Application Publication
(10) Pub. No.: US 2008/0299621 A1
(43) Pub. Date: Dec. 4, 2008

(54) MINIPREP SYSTEM FOR SIMPLE AND RAPID PLASMID DNA EXTRACTION

(75) Inventors: PETER JAMES TATNELL, Caerphilly (GB); DAVID WILLIAMS, Barry (GB)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE UK LIMITED, Buckinghamshire (GB)

(21) Appl. No.: 12/128,840

(22) Filed: May 29, 2008

Related U.S. Application Data
(60) Provisional application No. 60/941,075, filed on May 31, 2007.

(51) Int. Cl. C12P 19/34; C12M 1/00

(52) U.S. Cl. 435/91.1; 435/306.1

(57) ABSTRACT

The invention relates to a modified microspin system for the isolation and purification of plasmid DNA. A disposable pre-filter column is used in combination with the traditional microspin column for increase speed and quality of plasmid DNA preparation. The disposable pre-filter column includes a pre-filter and a depth filter for optimal result. During plasmid DNA isolation, the lysate can be loaded directly to the assembly including the disposable pre-filter column and the microspin column. A quick spin causes the plasmid DNA to bind to the microspin column while the flocculents remain on top of the disposable pre-filter column, eliminates the need to first remove the flocculants containing cellular debris. This results in a much shortened process. Also provided are kits for isolation of plasmid DNA including the pre-filter column and the microspin columns.

### CROSS-REFERENCE TO RELATED APPLICATIONS

This application claims priority to U.S. provisional patent application No. 60/941,075 filed on 31 May 2007; the disclosure of which is incorporated herein by reference in its entirety.

### FIELD OF THE INVENTION

This invention relates to an improved system and method for nucleic acid purification. More specifically, it relates to a simple and rapid system and method for the mini-scale extraction and purification of plasmid DNA from cells.

### BACKGROUND OF THE INVENTION

Plasmids are double-stranded supercoiled DNA molecules that range in size from 1 kb to more than 200 kb. Plasmids are useful tools in genetic engineering. They are widely used as vectors to carry foreign DNA; such that the foreign DNA is amplified and isolated or expressed. Plasmid DNA has also been utilized in the development of vaccines and in gene therapy.

The analysis and in vitro manipulation of plasmid DNA is typically preceded by an isolation step in order to free the nucleic acid from unwanted cellular contaminants which may interfere with subsequent processing procedures. A mini-scale sample preparation from an overnight bacterial culture of 1-5 ml generates more than enough plasmid DNA (a few micrograms) for these applications.

The most common mini-scale plasmid DNA extraction protocols exploit reagents originally developed by Birnboim and Doly (Birnboim, H. C. and Doly, J., Nucl. Acids Res. 7, 1513 (1979)), to separate supercoiled plasmid DNA from bacterial genomic DNA, RNA and protein. These reagents, developed many years ago, work on the principle of sequential use of three buffers, commonly referred to as buffer I, II and III. They each have distinct compositions to bring about plasmid enrichment and separation from contaminants. Buffer I is used to resuspend the bacterial pellet obtained by an initial centrifugation step of an appropriate bacterial culture. Once resuspended, buffer II is added which contains SDS detergent and NaOH. These components lyse the bacteria and denature the genomic DNA (pH>12). Buffer III typically contains a chaotrope to further denature protein, the chaotrope also promotes binding of plasmid DNA to the silica matrix commonly used in mini-prep columns. Buffer III also usually contains potassium acetate to rapidly neutralize the combined solutions. The addition of buffer III causes contaminants to “crash-out” of solution owing to the formation of insoluble complexes driven by rapid re-naturation of genomic DNA and the potassium salt of the detergent.

The insoluble flocculant material has traditionally been removed by a 5-10 minute centrifugation to “pack” the flocculant material at the bottom and side of a microfuge tube (See, e.g. ILLUSTRA™ plasmidPrep Mini Spin Kit, GE Healthcare, Piscataway, N.J.). The clarified plasmid-containing solution is subjected to a chromatographic separation. For mini-scale purification, the clarified solution is usually applied to a microspin column containing a glass fiber matrix or silica membrane. Plasmid DNA binds to the column in the presence of a chaotrope, while soluble impurities do not bind. After the soluble impurities are washed off, the plasmid DNA are eluted with an appropriate elution buffer.

It is advantageous to further simplify the process therefore to provide a more efficient mini-scale plasmid DNA purification method.

### SUMMARY OF THE INVENTION

In general, the instant invention provides improved methods, systems and kits for rapid isolation of plasmid DNA from plasmid containing cells in a mini-scale format.

In one aspect, the invention features a method for the rapid isolation of plasmid DNA, including: a) collecting plasmid-containing cells and resuspending them in an aqueous buffer; b) incubating the resultant mixture with a lysis/denaturation solution to lyse the cells and denature DNA; c) neutralizing the mixture with a renaturation solution to generate a renatured mixture of dissolved plasmid DNA and flocculants containing insoluble genomic DNA and cellular debris; d) loading the renatured mixture directly, without first removing said flocculants from the mixture, to a disposable column pre-assembled on top of a microspin column, which disposable column having both a pre-filter and a depth filter; e) passing loaded sample mixture through the assembly of disposable column and microspin column such that the flocculants are packed on top of the disposable column while plasmid DNA binds to microspin column matrix; f) washing the microspin column with a wash solution to remove soluble impurities after discarding said disposable top column; and g) eluting plasmid DNA from the microspin column with an elution buffer. It has been found surprisingly that by introducing a disposable pre-filter column, there is no longer a need to remove flocculants containing cellular debris by a time consuming centrifugation step, prior to loading the microspin column. Instead, the flocculants stay on top of the pre-filter column and is discarded, and do not interfere with subsequent wash or elution of the plasmid DNA.

In a second aspect, the invention provides a modified, disposable column for the rapid isolation of plasmid DNA from plasmid-containing cells, for use with the microspin column. The disposable column comprises a pre-filter and a depth filter. Preferably, the disposable column contains a glass fiber matrix or absorbent paper depth filter and the pre-filter and support filters are made of porous sintered polyethylene or polypropylene.

In another aspect, the invention provides kits for rapidly isolating plasmid DNA, including a modified, disposable column, a microspin column, reagents and user manual. During plasmid DNA purification, the disposable column is assembled on top of the microspin column and enables direct loading of lysate without first removal of the flocculants.

Certain aspects of the invention allow simultaneous isolation of a large number of different plasmids. The modified, disposable columns can be joined together to take the form of a microtiter plate. By this kind of an arrangement, a number of different plasmid containing cell cultures can be processed simultaneously. It is noted that all centrifugation steps can be replaced with vacuum.

The above and further features and advantages of the instant invention will become clearer from the following detailed description and claims.

### BRIEF DESCRIPTION OF THE DRAWINGS

FIG. 1 shows a schematic diagram of the modified microspin system according to one embodiment of the invention. A disposable column containing both a pre-filter and a depth filter is assembled on top of a traditional microspin column to capture the denatured flocculent material.

FIG. 2 shows a gel image of plasmid DNA isolated using the modified miniprep systems according to Example 2. The numbers represent either controls or a particular combination according to Table 1.

FIG. 3 shows the yield of plasmid DNA isolated according to the modified miniprep systems according to Example 3. The pre-filter and depth filters used are described in detail in Example 3.

FIG. 4 shows a gel image of plasmid DNA isolated using the modified miniprep systems according to the scheme of Example 3.

FIG. 5 shows a gel image of plasmid DNA of FIG. 4, after digestion with HindIII restriction enzyme.

### DETAILED DESCRIPTION OF THE INVENTION

The invention features improved processes, systems and kits for rapidly isolating plasmid DNA from plasmid containing cells, in particular for downstream applications in molecular biological research, such as cloning and sequencing. As used herein, the term “plasmid” refers to supercoiled DNA molecules (single or double stranded) that are maintained in a host cell separate from the host cell genome. Plasmids can be of a high copy number or low copy number and can carry any gene or external piece of DNA, either genomic or synthetic, encoding protein or peptide of interest, from any source.

In general, the improved process for isolating plasmid DNA includes the use of a pre-filter column in combination with the microspin column such that it eliminates the need to remove by centrifugation, the insoluble flocculant cellular debris generated from the lysis of the cells, before loading the column, thus simplifies the work flow and shortens the protocol significantly. A microspin column for plasmid DNA isolation generally contains a separation matrix placed on and supported physically by a disc of porous material more commonly referred to as a frit, typically made of sintered polyethylene. The holes so formed during the production of the frit material allow the unhindered passage of aqueous solutions and more importantly aqueous solutions containing plasmid DNA. The frit material is inert and does not interact to any great extent with DNA. The separation matrix is preferably a glass fiber matrix or a silica membrane. Alternatively, the matrix is a zeolite, or an organic matrix such as a resin or polymer.

One embodiment of the invention includes the use of a disposable prefilter column containing a pre-filter as well as a depth filter. One example of a pre-filter is a porous sintered polyethylene or polypropylene, similar to the support frit underneath the matrix of the microspin column. An example of a depth filter is a glass fiber matrix.

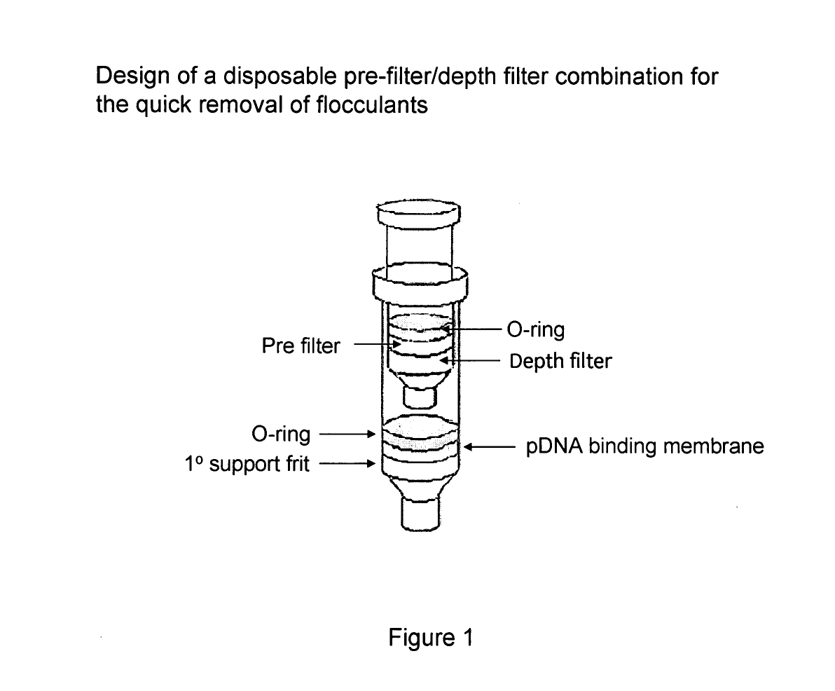

The pre-filter in the disposable column does not have to be a porous sintered polyethylene or polypropylene and indeed an optimal column might be composed of alternative materials having different filtration/binding characteristics. A thinner “pre-filter” material (e.g., cellulose absorbent paper or polypropylene mesh) may allow improved assembly of the column where sheets of appropriate components are layered together prior to die-cutting and positioning within a column moulding. Optionally, an O-ring can be used to secure the “pre-filter” (FIG. 1).

A depth filter is included between the pre-filter and the support disc (FIG. 1). A combination of both a pre-filter and a depth filter reduces residual contaminant flow-through from the pre-filter, thus is preferable for certain applications. A suitable depth filter is a glass microfiber filter or a cellulose paper. A preferred depth filter is one that captures any residual flow-through contaminants from the pre-filter yet retains minimum amount of plasmid DNA during elution.

Plasmids isolated in accordance with the invention can be of any origin. Most commonly, microorganisms like bacteria, such as E. coli, are used for culturing the plasmids, but the use of host cells is not limited and can be prokaryotic or eukaryotic cells. The host cells harboring the plasmid can be cultivated in a number of ways well known in the art, e.g. in incubator, bioreactor, fermentor etc. The plasmid isolated according to the invention can be of virtually any size, e.g. in the range of about 1 kb up to about 20 kb. As an upper limit, the isolation of cosmids and artificial chromosomes is also encompassed, the size of which may be up to about 50 kb and 500 kb, respectively.

The modified microspin column is suitable for extraction of plasmid DNA from standard cultures of bacteria. The inclusion of a disposable pre-filter column with a microspin column eliminates the need to centrifuge and remove flocculant material generated by alkaline lysis, prior to addition of sample to the DNA-binding column. Traditionally, lysate is clarified by a 5-10 minute spin in a micro-centrifuge before addition of the clarified lysate to the microspin column. Using the modified system, the most time consuming step is removed from the process without affecting quality of isolated product. Purification of plasmid DNA with the modified system can now be done in 5-6 minutes, compared to traditional process which typically takes about 10-20 minutes to complete.

During experimentation it was found that the use of the pre-filter column with microspin column in combination with a fixed-angle microcentrifuge enabled the insoluble flocculent material to be pelleted “over to one side” so that occlusion of the frit pre-filter was less likely to occur. Even without a fixed-angle rotor, using a vacuum that distributes flocculant material across the entire surface of the pre-filter, good quality plasmid DNA can still be obtained that can be digested and sequenced.

By deploying the modified miniprep system of a disposable prefilter column and the microspin column, it has been possible to achieve multiple benefits. First, it enables the addition of lysed sample to the modified column without centrifugation and removal of flocculants (pre-processing). It provides an improvement in the ease of use and time for completion, speeding up the process by more than 50%.

Methods for isolating plasmid DNA generally starts from culturing the host cells containing the plasmid. When the culture is ready, the cells are recovered by e.g. centrifugation or filtration. The cells can be stored, for example in a freezer, or processed immediately. The process for isolating plasmid DNA includes first collecting plasmid-containing cells and resuspending them in an aqueous buffer; then incubating with a lysis/denaturation solution to lyse the cells and denature DNA; followed by neutralizing the mixture with a renaturation solution to generate a renatured mixture of dissolved plasmid DNA and flocculants containing insoluble genomic DNA and cellular debris. In one aspect, the improved method includes loading the renatured solution with the flocculants directly to a disposable pre-filter column pre-assembled on top of a microspin column. The solution is then passed through the pre-filter column and the microspin column by centrifugation or vacuum, such that the flocculants are packed on top of the pre-filter column while plasmid DNA binds to microspin column matrix. The disposable column with the flocculants is discarded, and the microspin column is washed with a wash solution to remove soluble impurities. Plasmid DNA is eluted from the microspin column with an elution buffer. It is surprisingly discovered that although the flocculants are not removed by a centrifugation step, high quality plasmid DNA is isolated that is suitable for subsequent molecular biology analysis.

The protocols for cell lysis and denaturation of cellular debris are well known. A particularly useful aqueous buffer for resuspending plasmid-containing cells contains an isotonic buffer (e.g. a Tris buffer; or a sucrose or glucose solution), a chelating agent (e.g. ethylenediaminetetraacetic acid (EDTA) or (CDTA)) and an RNAse. This buffer may also optionally include lysozyme to further weaken cell walls. After the cells are resuspended, the cells are lysed and linear DNA is denatured, preferably by incubation in an alkaline lysis solution. Thorough lysis and denaturation can be accomplished by mixing the resuspended cells with a sodium hydroxide, sodium dodecyl sulfate solution. A third, renaturation solution (e.g. an acetate buffered solution, containing a chaotropic salt) is then added to yield a mixture containing plasmid DNA, insoluble clots of linear DNA and cellular debris.

According to one aspect of the invention, the renatured mixture of dissolved plasmid DNA and insoluble flocculants are loaded on to the disposable pre-filter column and placed in a microspin column. Through vacuum or centrifugation, liquids in the mixture passes through the columns, leaving on top of the pre-filter column a packed layer of flocculants, in the meantime plasmid DNA binds to microspin column matrix. The disposable pre-filter is then discarded. A wash solution is then applied to the microspin column to remove soluble impurities; and plasmid DNA is eluted from the microspin column with an elution buffer. The centrifugation step to remove the flocculants is eliminated and this does not affect the quality of the plasmid DNA isolated.

Certain aspects of the invention allow simultaneous isolation of a large number of different plasmids. The disposable pre-filter column, as well as the microspin columns can be joined together to take the form of a microtiter plate. Especially preferred are microtiter plates in the 96 well format. By this kind of an arrangement, a large number of plasmid containing cultures can be processed simultaneously. It is noted that all centrifugation steps can be replaced with vacuum.

### EXAMPLES

The following examples serve to illustrate the plasmid DNA purification processes according to embodiments of the present invention and are not intended to be limiting.

1. The protocol

The protocol is suitable for the rapid extraction and purification of plasmid DNA from 1.5 ml cultures of Escherichia coli (E. coli). The procedure can be completed in 5 to 6 minutes to yield plasmid DNA with a purity and quality compatible with many common molecular biology techniques, including cloning, restriction enzyme digestion, PCR amplification and DNA sequencing.

The plasmid DNA yield from a freshly grown E. coli strain containing a high copy number plasmid (>300 copies/cell) and grown to A₆₀₀ approximately 2.5 is typically 3 to 6 μg (A₂₆₀/A₂₈₀>1.8).

The protocol utilizes a simple plasmid DNA purification process, employing a modified alkaline cell lysis procedure and a silica-based membrane. No organic solvents are used; instead, chaotropic salts are included to denature protein components and promote the selective binding of plasmid DNA to the silica membrane. Denatured insoluble contaminants are retained on top of disposable pre-filter column, while soluble contaminants are easily removed by subsequent washing. The purified plasmid DNA is eluted in a low ionic strength buffer, at a plasmid concentration suitable for most molecular biological applications.

The following provides a step by step protocol:
1. Transfer 1.5 ml from a fresh overnight culture to a microcentrifuge tube. To pellet bacteria, centrifuge (13 000×g) for 30 seconds. Discard supernatant and re-centrifuge. Remove any residual supernatant using a pipette.
2. Thoroughly re-suspend the pellet by adding 175 μl lysis buffer (100 mM Tris-Cl pH7.5; 10 mM EDTA; 0.4mg/ml RNase I), and either vortexing, pipetting up and down or scraping the base of the microcentrifuge tube across the surface of an empty pipette tip rack.
3. Cell lysis—Add 175 μl lysis buffer (200 mM NaOH; 1% SDS) and mix immediately by gentle inversion (approximately 5 times) until solution becomes clear and viscous.
4. Neutralisation—Add 350 μl neutralization buffer (4.4M Guanidine HCl, 0.65M potassium Acetate and 3.1M Glacial Acetic Acid), and mix immediately by gentle inversion until the precipitate is evenly dispersed.
5. Transfer the neutralized mixture (approximately 600 μl) to a pre-filter column already placed on top of a microspin column (FIG. 1). Close the lid of the column gently. Centrifuge (13 000×g) for 30 seconds. Discard the pre-filter column. Discard the flow through by emptying the collection tube.
6. Wash the microspin column with 400 μl wash buffer (2mM Tris-Cl pH8; 0.2mM EDTA and 80% ethanol) and centrifuge (13 000×g) for 30 seconds. Discard the flow-through and repeat the wash one more time with a 60 second spin.
7. Move the microspin column into a fresh microcentrifuge tube and add 100 μl elution buffer (10 mM Tris-Cl pH8) directly onto the centre of the column. Incubate the column for 30 seconds at room temperature. Microcentrifuge (2 000×g) for 60 seconds to recover the plasmid DNA as flow through in the microcentrifuge tube.

Purified plasmid DNA concentration should be determined by UV spectrophotometry (A₂₆₀) and through comparison with a known standard by agarose gel electrophoresis and subsequent densitometric analysis. If available, the UV spectrophotometric ratios A₂₆₀:A₂₈₀ and A₂₆₀:A₂₃₀ provide a limited indication of purity as measures of protein and salt contamination.

2. Purification of plasmid DNA—evaluation of pre-filters

To reduce extraction time, the use of a disposable pre-filter column was tested on a traditional microspin column (FIG. 1). Gross (denatured gDNA/protein etc) and fine (precipitated KDS) components of the flocculent were retained by the disposable filter column and thereby prevented access to the microspin column. A single wash of the microspin column was sufficient to generate quality plasmid DNA therefore reducing the time required to isolate plasmid DNA to about 5.5 minutes.

The above protocol from Section 1 was used for plasmid DNA isolation. Absorbance data was determined using a Nanodrop ND 1000 spectrophotometer.

A number of pre-filter column combinations were tested, together with a silica membrane microspin column (ILLUSTRA™ plasmidPrep Mini Spin kit). To compare the quality and yield with traditional protocols, controls were included. The control experiments were performed following manufacturer's protocols. The pre-filter column combinations generally included a pre-filter and a depth filter. The depth filter used in these experiments was the Whatman GF/B glass microfibre depth filter. Table 1 lists the pre-filters tested in combination with this depth filter, as well as the control experiments performed.

TABLE 1
Pre-filter columns - pre-filters tested with the glass microfibre depth filter.

| | | Depth |
|---|---|---|
| | Control or Pre-filter composition | filter |
| 1 | Control - Qiagen QIAPREP ™ Spin Mini kit (Cat. | no |
| | 27106) | |
| 2 | Control - ILLUSTRA ™ plasmidPrep Mini Spin kit (Cat. | no |
| | 28-9042-69) | |
| 3 | Pall Corp. LAS (40 um) (P/N 0270550201) | yes |
| 4 | Pall Corp. Accuwik Ultra (P/N SPR0730) pre-filter | yes |
| 5 | Pall Corp. Absorbent Paper 165 (P/N S70009) | yes |
| 6 | Handee Spin column PE frit (25 um) (P/N 89868) | yes |
| 7 | Porex X-4920 PE frit (10-20 um pore size) | yes |
| 8 | Novagen Clear Filter Spin (Cat 708483) | yes |
| 9 | Macherey Nagel 96-well mesh | yes |
| 10 | Macherey Nagel QUICKPURE ™ column PE frit (Cat | yes |
| | 406184) | |

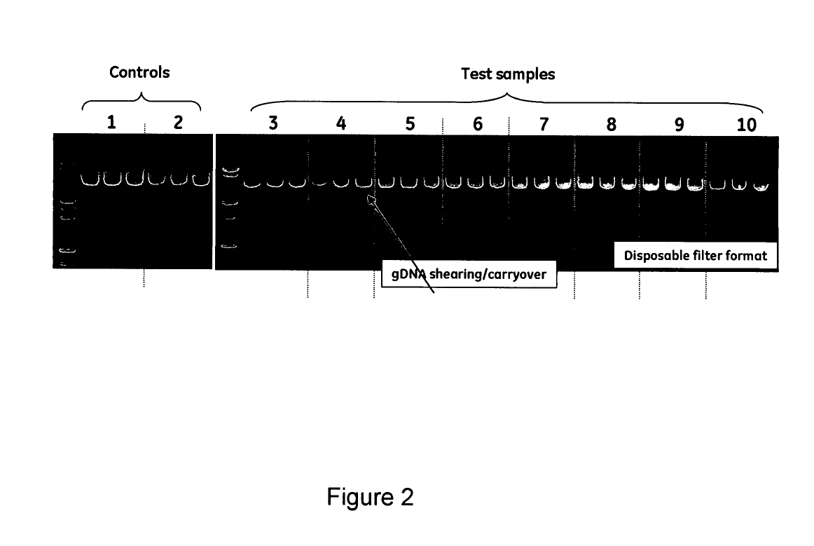

For each pre-filter/depth filter combination (or control experiment), at least three parallel experiments were run. It was found that with a disposable pre-filter column, the time needed to complete a plasmid DNA isolation experiment was about 5.5 min. In comparison, the ILLUSTRA™ plasmidPrep Mini Spin kit took about 9 min to complete, while the QIAPREP™ Spin Mini kit took about 19 min to complete. In general, the modified system generated plasmid DNA of comparable quality to those isolated using the control kits. The low level of protein contaminants was similar. The amount of particulates in the final elution was marginally lower compared to control extractions. Majority of uncut plasmid DNA was in the supercoiled configuration (FIG. 2). Therefore in general the quality of the isolated plasmid DNA was comparable to control extractions.

It was noted that salt levels in the eluted DNA using the disposable pre-filter column were significantly lower and therefore better than that of the controls. The amount of plasmid DNA isolated was about 20-60% that of the controls. This was expected, as the depth filter used here in the disposable filters was a glass microfibre matrix which could bind plasmid DNA. However, the amount of plasmid DNA isolated was enough for routine downstream manipulations and was of high quality.

The use of a disposable pre-filter column in addition to the traditional microspin column increased the speed from a traditional microspin column with yet better quality compared with traditional spin extraction methods/kits as evidence by reduced salt levels. Even though the micro-fibre depth filter bound some plasmid DNA in the presence of the chaotrope, the yield of the plasmid DNA was sufficient for routine downstream molecular biology manipulations.

3. Purification of plasmid DNA—evaluation of depth filters

We further tested the performance of additional pre-filter and depth filter combinations for the isolation of plasmid DNA, together with the traditional microspin column. The pre-filters tested include:
Porex: Porex X-4920 10-20 um (PE)
Handee: Handee spin column frit (89869, HH106259)
MN frit: Macherey Nagel QUICKPURE™ column PE frit (Cat 406184)

The depth filters tested include:
113: Pall Absorbent Paper 113—P/N S70007, lot 20410
133: Pall Absorbent Paper 133—P/N S70008, lot 64515
165: Pall Absorbent Paper 165—P/N S70009, lot 70301
GFB: Whatman GF/B glass microfibre depth filter

To compare the quality and yield with traditional protocols, ILLUSTRA™ plasmidPrep Mini Spin kit was used as a control. The control experiment was performed following manufacturer's protocol

For each pre-filter/depth filter combination (or control experiment), at least two parallel experiments were run. In general, the modified system generated plasmid DNA of comparable quality to those isolated using the control kits. Flocculants were retained on top of the pre-filters, yet the disposable columns all exhibited good flow characteristics. Minor quantities of KDS were observed in the flow through of the pre-filter columns containing Pall absorbent paper depth filters, but not in the pre-filter columns with the Whatman GF/B glass microfibre depth filter. This slightly higher salt levels in some of the minipreps, however, does not appear to be a problem for downstream applications as evidence by subsequent analysis of the isolated plasmid DNA.

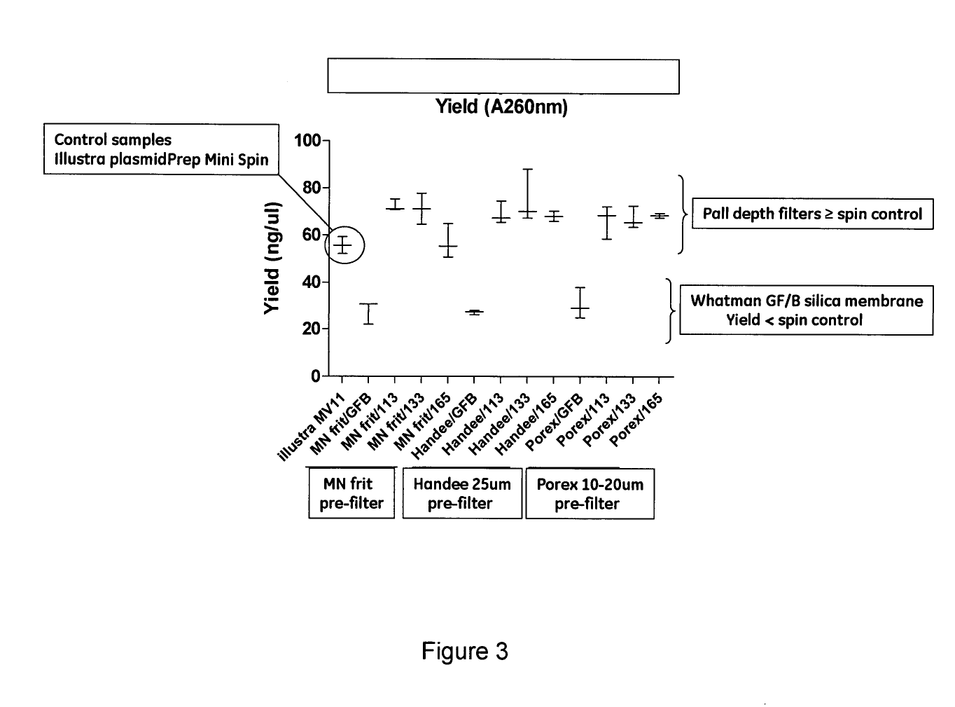

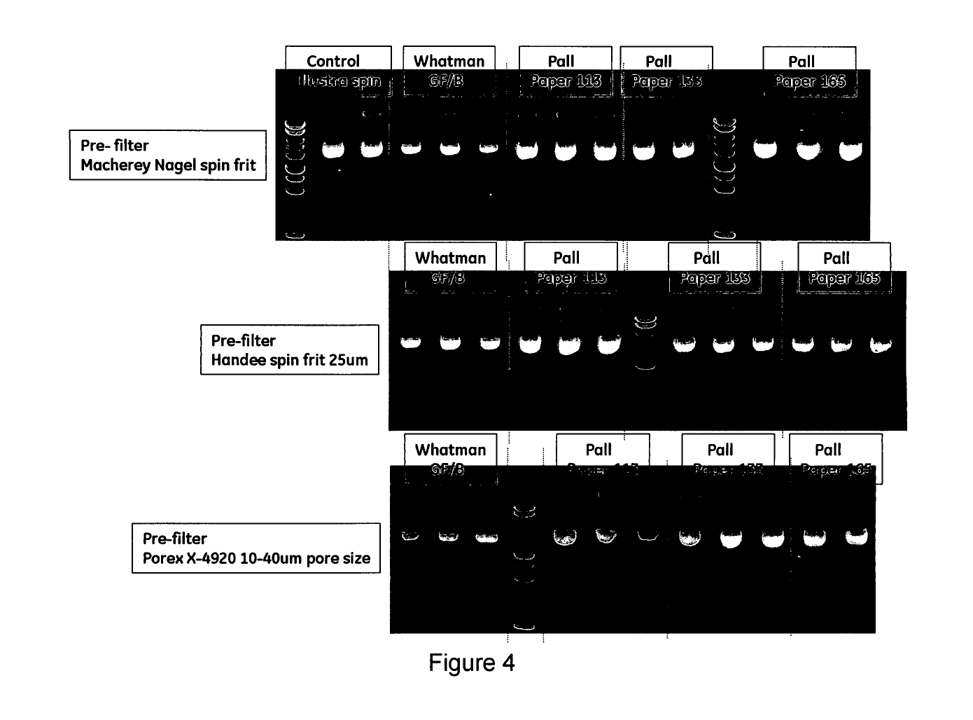

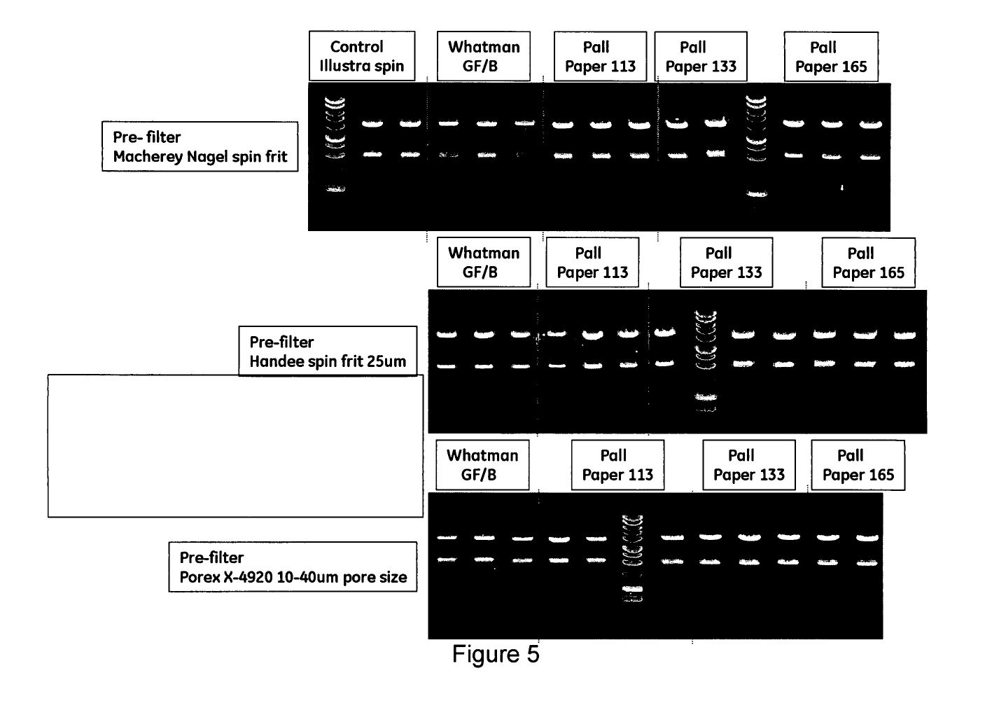

Low levels of protein contaminants was observed. The amount of particulates in the final elution was equivalent to control extractions. The miniprep performed with pre-filter columns with the Pall absorbent paper depth filter has a higher yield in general, as compared to the minipreps using a pre-filter column with the Whatman GF/B glass microfibre depth filter (FIG. 3). This was expected, as the Whatman GF/B glass microfibre depth filter used could bind plasmid DNA. Majority of uncut plasmid DNA was in the supercoiled configuration (FIG. 4), although some genomic DNA was observed in the minipreps isolated with the pre-column containing the Pall absorbent paper depth filter. Restriction digest of the plasmid DNA was successful for all the samples isolated (FIG. 5), proving the quality of the plasmid DNA. Therefore in general the quality of the isolated plasmid DNA was comparable to control extractions.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. A method for the rapid isolation of plasmid DNA from plasmid-containing cells, including:
a) collecting said plasmid-containing cells and resuspending them in an aqueous buffer;
b) incubating the resultant mixture with a lysis/denaturation solution to lyse the cells and denature DNA;
c) neutralizing the mixture from step b) with a renaturation solution to generate a renatured mixture of dissolved plasmid DNA and flocculants containing insoluble genomic DNA and cellular debris;
d) loading the renatured mixture from step c) directly, without first removing said flocculants from the mixture, to a disposable column preassembled on top of a microspin column, said disposable column includes both a pre-filter and a depth filter;
e) passing loaded sample mixture through the assembly of disposable column and microspin column such that flocculants are packed on top of said disposable column while plasmid DNA binds to microspin column matrix;
f) washing said microspin column with a wash solution to remove soluble impurities after discarding said disposable top column; and
g) eluting plasmid DNA from said microspin column with an elution buffer.

2. The method of claim 1, wherein the depth filter in said disposable column includes a glass fiber matrix.

3. The method of claim 1, wherein said pre-filter in said disposable column is a disc of porous, sintered polyethylene or polypropylene.

4. The method of claim 1, wherein each of steps e) through g) is assisted by vacuum.

5. The method of claim 1, wherein each of steps e) through g) is assisted by centrifugation.

6. The method of claim 1, wherein the aqueous buffer in step a) is comprised of an isotonic buffer, a chelating agent and an RNAse.

7. The method of claim 6, wherein the isotonic buffer is selected from the group consisting of: a Tris buffer, a sucrose solution or a glucose solution.

8. The method of claim 7, wherein the chelating agent is selected from the group consisting of EDTA and CDTA.

9. The method of claim 8, wherein the aqueous buffer additionally comprises a lysozyme.

10. The method of claim 1, wherein the lysis solution is alkaline.

11. The method of claim 10, wherein the lysis solution comprises sodium hydroxide and sodium dodecyl sulfate.

12. A modified miniprep system for the preparation of plasmid DNA, comprising (a) a disposable column including both a pre-filter and a depth filter; and (b) a microspin column having a matrix that selectively binds plasmid DNA.

13. The modified microspin column of claim 12, wherein the matrix in the microspin column is a glass fiber matrix, a silica membrane or a zeolite.

14. The modified microspin column of claim 12, wherein the depth filter in said disposable column includes a glass fiber matrix.

15. The modified microspin column of claim 12, wherein the pre-filter in said disposable column is a disc of porous, sintered polyethylene or polypropylene.
